(19) United States
(12) Patent Application Publication
(10) Pub. No.: US 2011/0237832 A1
(43) Pub. Date: Sep. 29, 2011

(54) SYNTHESIS OF HDAC INHIBITORS: TRICHOSTATIN A AND ANALOGUES

(75) Inventors: Paul Helquist, Granger, IN (US); Douglas Schauer, Lafayette, IN (US); Casey C. Cosner, Mishawaka, IN (US); Vijaya B.R. Iska, Jaladanki (IN); Anamitra Chatterjee, Purulia (IN); Olaf Wiest, South Bend (IN)

(73) Assignee: University of Notre Dame du Lac, Notre Dame, IN (US)

(21) Appl. No.: 13/028,683

(22) Filed: Feb. 16, 2011

Related U.S. Application Data
(60) Provisional application No. 61/341,296, filed on Mar. 29, 2010.
(60) Provisional application No. 61/400,435, filed on Jul. 28, 2010.

(51) Int. Cl. C07C 259/06

(52) U.S. Cl. 562/623

(57) ABSTRACT

Embodiments herein relate to histone deacetylaces (HDACs) and HDAC inhibitors, such as trichostatin A (TSA) and TSA analogues. Embodiments provide simple methods of synthesizing TSA and TSA analogues. These methods provide routes of synthesis of TSA and TSA analogues that enable the production of the HDAC inhibitors at lower cost and in greater quantities than previously were available.

### CROSS REFERENCE TO RELATED APPLICATIONS

The present application is a nonprovisional of and claims priority to U.S. Provisional Patent Application No. 61/341,296, filed Mar. 29, 2010, entitled “Cost-Effective and Scalable Synthesis of HDAC Inhibitors: Trichostatin A and Analogues,” and U.S. Provisional Patent Application No. 61/400,435, filed Jul. 27, 2010, entitled “Synthesis of HDAC Inhibitors: Trichostatin A and Analogues,” the disclosures of which are hereby incorporated by reference in their entirety.

The present application is also related to U.S. patent application Ser. No. 12/888,267, filed Sep. 22, 2010, entitled “HISTONE DEACETYLASE INHIBITORS AS THERAPEUTIC AGENTS FOR LYSOSOMAL STORAGE DISORDERS,” the disclosure of which is hereby incorporated by reference in its entirety.

### GOVERNMENT INTERESTS

This invention was made with Government support under Grant No. CHE-0833220 awarded by the National Science Foundation. The Government has certain rights in the invention.

### TECHNICAL FIELD

Embodiments herein relate to histone deacetylaces (HDACs) and HDAC inhibitors, for example trichostatin A (TSA) and TSA analogues, and more specifically, to methods of synthesizing TSA and TSA analogues.

### BACKGROUND

TSA is one of the most potent HDAC inhibitors available. In addition to its anti-fungal, antibiotic, and anti-malarial activities, TSA arrests cell cycle progression in G1 and inhibits the activity of HDACs with an IC₅₀ value of 70 nM in human T cells, shows anti-cancer activity by slowing the progression of cancer through gene expression, and inhibits the accumulation of cholesterol in some cell lines. As described in U.S. patent application Ser. No. 12/888,267, TSA may be used for the treatment of lysosomal storage disorders such as Niemann-Pick type C disease.

Unfortunately, the current cost of TSA is exorbitant, and limits on natural sources have become bottlenecks for its further development as a therapeutic agent. Although a few reports for the preparation of this compound have been published, none of them provides a reliable procedure for its gramscale preparation.

### DETAILED DESCRIPTION OF DISCLOSED EMBODIMENTS

In the following detailed description, reference is made to the accompanying drawings which form a part hereof, and in which are shown by way of illustration embodiments that may be practiced. It is to be understood that other embodiments may be utilized and structural or logical changes may be made without departing from the scope. Therefore, the following detailed description is not to be taken in a limiting sense, and the scope of embodiments is defined by the appended claims and their equivalents.

Various operations may be described as multiple discrete operations in turn, in a manner that may be helpful in understanding embodiments; however, the order of description should not be construed to imply that these operations are order dependent.

The description may use perspective-based descriptions such as up/down, back/front, and top/bottom. Such descriptions are merely used to facilitate the discussion and are not intended to restrict the application of disclosed embodiments.

For the purposes of the description, a phrase in the form “A/B” or in the form “A and/or B” means (A), (B), or (A and B). For the purposes of the description, a phrase in the form “at least one of A, B, and C” means (A), (B), (C), (A and B), (A and C), (B and C), or (A, B and C). For the purposes of the description, a phrase in the form “(A)B” means (B) or (AB) that is, A is an optional element.

The description may use the terms “embodiment” or “embodiments,” which may each refer to one or more of the same or different embodiments. Furthermore, the terms “comprising,” “including,” “having,” and the like, as used with respect to embodiments, are synonymous.

As used herein, the formulas used in the specification and/or claims may represent a single compound, a mixture of compounds, a single enantiomer or a mixture of enantiomers (e.g., a racemic mixture), a single diastereomer, or a mixture of diastereomers, etc., unless otherwise specified.

For the purposes of the present disclosure, the following abbreviations are used in the specification, in the claims or in the drawing figures: “Bn” refers to benzyl, “Me” refers to methyl, “Et” refers to ethyl, “TFA” refers to trifluoroacetic acid, “TSA” refers to trichostatin A, and “MeOH” refers to methanol.

As used herein, the term “intermediate” typically refers to a compound or compounds that are prepared by a process or step of the present disclosure that is a precursor of, and can be subsequently used, directly or indirectly, to prepare an end product. For example, intermediates may be used to prepare other intermediates that are then used to prepare an end product.

As used herein, the term “end product” refers to the product obtained at the end or completion of the process, and is typically the product that is ultimately desired from the process.

As used herein, the term “process” refers to one or more steps used to prepare one or more compounds, including one or more intermediates, as well as one or more end products.

As used herein, the term “scheme” refers to a synthesis design, framework, etc., comprising two or more processing steps for preparing specific intermediates and/or end products.

As used herein, the term “Marshall coupling” refers to a palladium-catalyzed reaction of a propargyl sulfonate with a carbonyl compound to provide a homopropargyl alcohol.

As used herein, the term “mesylate” refers to a methane sulfonate.

As used herein, the term “TSA analogue” refers to a compound that includes one or more variations of substituents and/or functional groups without significantly changing the molecular skeleton of TSA, and that retains at least 5% of TSA's HDAC-inhibiting activity. In some embodiments, the term “TSA analogue” may refer to compounds having the following structure:

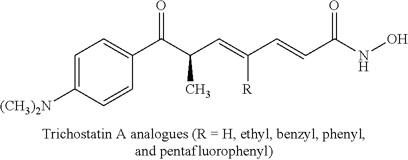

Disclosed herein in various embodiments are short and efficient methods for synthesizing TSA and TSA analogues. In various embodiments, these methods may allow the inexpensive production of TSA from simple and readily-available aromatic carbonyl compounds using metal-catalyzed reactions. In various embodiments, the methods may produce TSA in good yield in fewer steps than known synthetic pathways.

TSA contains one chiral center (R) with an array of two consecutive E double bonds.

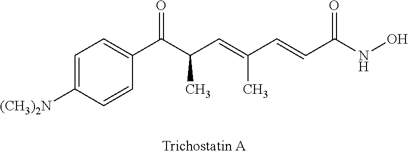

TSA also has a hydroxamic acid functionality on one end and a substituted aromatic group on the other. In various embodiments, as below in Scheme A, trichostatic acid, the direct precursor to TSA, may be prepared by a convergent approach wherein the chiral center is fixed by a palladium-catalyzed Marshall coupling of the aldehyde 1 with a chiral mesylate 2 containing a propyne moiety. In various embodiments, further manipulations of the resulting alkyne 3 may lead to a suitable alkenylboron intermediate that undergoes palladium-catalyzed Suzuki-Miyaura coupling with the other half of the molecule containing an unsaturated ester to provide 6. The coupling product readily provides trichostatic acid, which is a direct precursor to TSA, and one of skill in the art will know of methods of converting trichostatic acid into TSA. In some embodiments, the method may include a novel direct modification of the use of alkyne 5, which includes performing the Suzuki-Miyaura coupling reaction with a hydroxamic acid derivative 8.

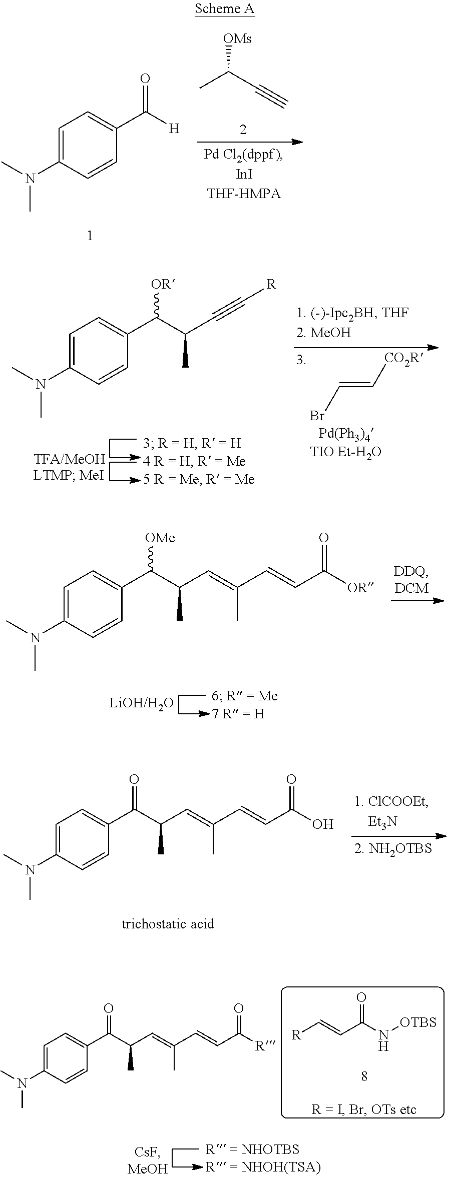

In this and the following examples, all reactions were performed under an inert atmosphere with stirring unless otherwise noted. Tetrahydrofuran and diethyl ether were purified using an Innovative Technologies™ solvent purification system. Anhydrous dichloromethane and methanol were purchased from Aldrich with sure seal septum. All reagents were used as purchased except where otherwise noted. Flash chromatography was performed using Silica 60A (230-400 mesh). ¹H NMR (300, 400 & 500 MHz) and ¹³C NMR (75, 100 & 125 MHz) spectra, were recorded on Varian Inova-300 & 500 spectrometers, and ¹H NMR (400 MHz) and ¹³C NMR (100 MHz) were recorded on a Bruker DPX-400 spectrometer. All ¹H NMR spectra were recorded in CDCl₃, or MeOD, and chemical shifts are given relative to CHCl₃ (7.27 ppm) or CD₃OD (3.34 & 4.87 ppm) and ¹³C NMR spectra are referenced to CDCl₃ (77.23 ppm) or CD₃OD (49.86 ppm). IR spectra were obtained with a Perkin-Elmer Paragon 1000 FT-IR spectrophotometer using neat thin films or CHCl₃ solutions between NaCl plates. Mass spectra were recorded on a JEOL JMS-AX505 HA double sector mass spectrometer.

In one specific, non-limiting example, the method illustrated in Scheme A may be carried out as follows:

### (2R)-1-(4-(dimethylamino)phenyl)-2-methylbut-3-yn-1-ol (3)

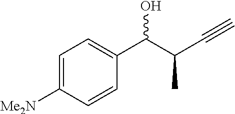

Based upon a Marshall procedure, PdCl₂(dppf) (307 mg, 0.42 mmol, 5 mol %) and Inl (4.055 g, 16.78 mmol) were added successively to a solution of (S)-but-3-yn-2-yl methanesulfonate (1.614 g, 10.9 mmol) and 4-N,N-dimethylamino benzaldehyde (1.25 g, 8.39 mmol) in dry THF (30 mL) and HMPA (6 mL) stirring at 0° C. The resulting dark green colored mixture was stirred for 1 hour, during which time the color turned brick red. The reaction mixture was quenched after 1 hour by the addition of water (50 mL), and was diluted with ether (50 mL). The organic layer was separated, and the aqueous layer was further washed with ether (3×50 mL). The combined organic layers were washed with brine, dried over anhydrous MgSO₄ and concentrated with under vacuum. The resulted crude mixture was purified using flash chromatography (hexane/EtOAc, 9/1) to get the title compound 3 as a 1:1 mixture of syn and anti diastereomers (1.413 g, 83%). The ¹H NMR spectrum was identical with previously reported for compound 3. ¹H NMR (400 MHz, CDCl₃): δ_{H} 7.31-7.20 (m, 4H), 6.78-6.69 (m, 4H), 4.65-60 (d, J=2.54 Hz, 1H), 4.46-4.40 (d, J=2.54 Hz, 1H), 2.96 (s, 12H), 2.90-2.82 (m, 1H), 2.82-2.75 (m, 1H), 2.50-2.48 (b, 2H), 2.22-2.20 (m, 1H), 2.11-2.09 (m, 1H), 1.20-1.14 (d, J=2.54 Hz, 3H), 1.11-1.06 (d, J=2.54 Hz, 3H).

### 4-((2R)-1-methoxy-2-methylbut-3-yn-1-yl)-N,N-dimethylaniline (4)

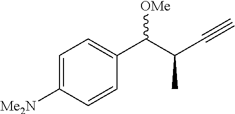

To a solution of compound 3 (1.310 g, 6.45 mmol) in MeOH (45 mL) was added a solution of 0.1% TFA in MeOH (v/v) (75 mL), and was stirred at 25° C. for 48 hours. The resulting dark brown solution was neutralized by careful addition of TEA, and all the volatile materials were removed under vacuum. The resulted crude mixture was purified using flash chromatography (hexane/EtOAc, 9/1) to get the title compound 4 as a 1:1 mixture of syn and anti diastereomers (1.315 g, 94%). The ¹H NMR spectrum was identical with that previously reported for compound 4. ¹H NMR (400 MHz, CDCl₃): δ_{H} 7.26-7.14 (m, 4H), 6.74 (d, J=8.7 Hz, 4H), 3.98 (d, J=2.54 Hz, 2H), 3.22 (S, 3H), 3.21 (S, 3H), 2.94 (S, 12H), 2.90-2.83 (m, 1H), 2.81-2.73 (m, 1H), 2.16 (d, J=2.4 Hz, 1H), 2.03 (d, J=2.4 Hz, 1H), 1.21 (d, J=6.9 Hz, 3H), 1.03 (d, J=6.9 Hz, 3H).

### 4-((2R)-1-methoxy-2-methylpent-3-yn-1-yl)-N,N-dimethylaniline (5)

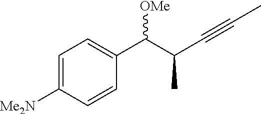

To a solution of compound 4 (1.272 g, 5.86 mmol) in dry THF (20 mL) was added LTMP (1.55 g in 20 mL of THF, 10.55 mmol) at −78° C. via cannula. After stirring for 30 minutes, MeI (0.44 ml, 7.03 mmol) was added at the same temperature. The resulting mixture was stirred for another 30 minutes at −78° C. before bringing to 0° C., at which temperature it was stirred for 6 hours. The reaction mixture was quenched by careful addition of saturated NH₄Cl solution (20 mL). The organic layer was separated and the aqueous layer was washed with ether (3×20 mL). The combined organic layers were dried over MgSO₄ and concentrated in vacuo. The crude mixture was purified by column chromatography (EtOAc/hexanes=1/4) to obtain compound 5 (1.326 g, 98%). The ¹H NMR spectrum was identical with the reported values for compound 5. ¹H NMR (300 MHz, CDCl₃): δ_{H} 7.23-7.15 (m, 4H), 6.77-6.69 (m, 4H), 3.94 (d, J=2.4 Hz, 2H), 3.92 (d, J=2.4 Hz, 2H), 3.23 (s, 6H), 2.97 (s, 12H), 2.83-2.63 (m, 2H), 1.83 (d, J=2.4 Hz, 3H), 1.74 (d, J=2.4 Hz, 3H), 1.14 (d, J=7.1 Hz, 3H), 1.00 (d, J=7.2 Hz, 3H).

### (2E,4E,6R)-methyl 7-(4-(dimethylamino)phenyl)-7-methoxy-6-methylhepta-2,4-dienoate (6)

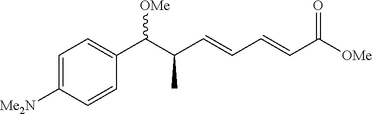

(−)-Ipc₂BH (1.943 g, 6.77 mmol) was weighed in a glove box into a round-bottom flask. The flask was placed in an ice bath and a solution of (+)-compound 5 (1.303 .g, 5.64 mmol) in THF (20 mL) was added. The mixture was stirred for 2 hours at 0° C., and then MeOH (0.53 mL, 13.39 mmol) was added. After 2 hours, a solution of (E)-methyl 3-bromoacrylate (1.388 g, 8.46 mmol) in THF (20 mL) was added to the resulting solution at 0° C., and the flask was allowed to warm to room temperature. To the solution were added Pd(PPh₃)₄ (652 mg, 0.56 mmol, 10 mol %) and TIOEt (1.2 mL, 16.9 mmol) in H₂O (12 mL). The resulting off-white colored mixture was stirred for 1 hour at ambient temperature, and then the mixture was diluted with 1 M aqueous NaHSO₄ (20 mL). The mixture was filtered and extracted with Et₂O (3×50 mL). The organic extracts were washed with brine, dried over MgSO₄, and concentrated. The crude product was purified by flash chromatography (hexanes/EtOAc=95/5 to 90/10) to give the coupling product 6 with some inseparable impurities. This crude product was directly used to the next step without any further purification.

### (2E,4E,6R)-7-(4-(dimethylamino)phenyl)-7-methoxy-6-methylhepta-2,4-dienoic Acid (7)

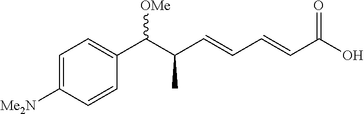

Compound 6 (1.609 g, 5.08 mmol) was dissolved in MeOH (40 mL) and treated with 0.5 M LiOH solution (13 mL, 6.1 mmol). The resulting solution was stirred at 45° C. for 24 hours. The mixture was then neutralized by the addition of pH-7 buffer. The volatile part was removed under vacuum, and the remaining yellow residue was washed with EtOAc (2×20 mL). The aqueous layer was acidified to pH-4 using 1N HCl and was then extracted with CHCl₃ (3×50 mL). The combined organic extracts were dried, and concentrated to get crude compound 7 as a yellowish syrup. The compound was purified by flash chromatography (hexanes/EtOAc=4/1 to 1/1) to obtain pure compound 7 (1.384 g, 81%). The ¹H NMR spectrum was identical with previous reports. ¹H NMR (300 MHz, CDCl₃): δ_{H} 7.45 (d, J=15.6 Hz, 1H), 7.31 (d, J=15.6 Hz, 1H), 7.19-7.08 (m, 4H), 6.76-6.65 (m, 4H), 5.94 (d, J=9.8 Hz, 1H), 5.82-5.70 (m, 3H), 3.95-3.89 (m, 2H), 3.20 (s, 3H), 3.18 (s, 3H), 2.98 (s, 6H), 2.96 (s, 6H), 2.94-2.83 (m, 2H), 1.74 (s, 3H), 1.63 (s, 3H), 1.09 (d, J=6.9 Hz, 3H), 0.86 (d, J=6.9 Hz, 3H).

### (R,2E,4E)-7-(4-(dimethylamino)phenyl)-6-methyl-7-oxohepta-2,4-dienoic Acid ((+)-Trichostatic Acid)

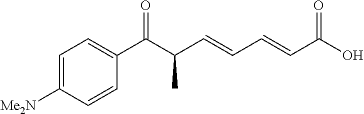

Compound 7 (1.295 g, 4.27 mmol) was dissolved in DCM/H₂O (50 mL, 2/1) and treated with DDQ (920 mg, 4.06 mmol) in 3 portions over a period of 5 minutes at 0° C. The resulting mixture was stirred vigorously for another 5 minutes at the same temperature, and was diluted by adding DCM (20 mL). The mixture was filtered through celite and washed with DCM (50 mL). The filtrate was dried over MgSO₄ and concentrated to obtain reddish brown trichostatic acid (1.067 g, 87%). The compound was found to be sufficiently pure by ¹H NMR to carry on to the next reaction. The ¹H NMR spectrum was identical with the literature data. [α]_{D}²⁵: +138° (c 0.095, EtOH); ¹H NMR (300 MHz, CDCl₃): δ_{H} 7.85 (d, J=9.0 Hz, 2H), 7.39 (d, J=15.6 Hz, 1H), 6.65 (d, J=9.0 Hz, 2H), 6.10 (d, J=9.6 Hz, 1H), 5.83 (d, J=15.6 Hz, 1H), 4.41 (dq, J=9.6, 6.6 Hz, 1H), 3.07 (s, 6H), 1.95 (s, 3H), 1.34 (d, J=6.6 Hz, 3H).

### (+)-Trichostatin A (7)

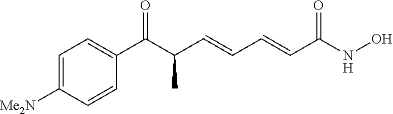

Trichostatic acid (1.015 g, 3.54 mmol) and TEA (1.1 mL, 7.78 mmol) were dissolved in DCM (20 mL) and cooled to 0° C. Chloroethyl formate (0.4 mL, 4.24 mmol) was added and the resulted solution was stirred at the same temperature for 2 hours, followed by the addition of NH₂OTBS (780 mg, 5.3 mmol). After stirring for 30 minutes, the cooling bath was removed, and the reaction mixture was allowed to warm to room temperature at which it was further stirred for 2 hours. The reaction mixture was then diluted with DCM (30 mL) and the organic layer was washed with water (20 mL). The aqueous layer was further extracted with DCM (3×30 mL). The combined organic layers were washed with brine, dried over anhydrous MgSO₄, and concentrated under vacuum to obtain crude protected hydroxamic acid, which was directly used in the next step without any further purification.

The above crude compound was dissolved in anhydrous MeOH (30 mL) and treated with dry CsF (645 mg, 4.24 mmol). The resulting mixture was stirred at room temperature for 3 hours and diluted with EtOAc (50 mL). The organic layer was washed with water (20 mL), and the aqueous layer was extracted with EtOAc (3×20 mL). The organic layers were combined, dried over MgSO₄, and concentrated under vacuum. The resulting solid mass was triturated with hexanes/Et₂O (4/1) solution to obtain pure (+)-trichostatin (983 mg, 92% in two steps). The ¹H NMR spectrum was identical with the literature data. [α]_{D}²⁵: +96° (c 0.13, EtOH); ¹H NMR (300 MHz, CD₃OD): δ_{H} 7.83 (d, J=9.0 Hz, 2H), 7.15 (d, J=15.6 Hz, 1H), 6.70 (d, J=9.0 Hz, 2H), 5.89 (d, J=9.6 Hz, 1H), 5.83 (d, J=15.6 Hz, 1H), 4.54 (dq, J=9.6, 6.6 Hz, 1H), 3.03 (s, 6H), 1.91 (d, J=1.2 Hz, 3H), 1.23 (d, J=6.6 Hz, 3H). IR (CHCl₃) v 3427, 3236, 2928, 1659, 1599, 1551, 1379, 1248, 1192, 1058, 972, 818 cm⁻1; HRMS (ESI) Calcd. For C₁₇H₂₃N₂O₃ [M+H]⁺: 303.1703 Found: 303.1722.

In other embodiments, a similar method to that illustrated in Scheme A may be used to produce novel analogues of trichostatic acid. In some embodiments, these may be further manipulated to form their corresponding TSA analogues, such as those represented by the following structure:

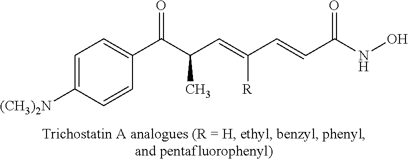

In various embodiments, straightforward modifications to Scheme A may be employed to yield other TSA analogues.

In various embodiments, hydrophobic interactions present in the 11 A channel of the active site may be used to produce analogs with hydrophobic groups introduced at the C-4 carbon of TSA. In some embodiments, the methyl group at C-4 may be transposed with hydrogen, ethyl, isopropyl, propyl, hydroxypropyl, t-butyl, butyl, phenyl, hydroxyphenyl, benzyl, or hydroxybenzyl.

As illustrated below in Scheme B, in various embodiments, the synthesis of C-4 ethyl, benzyl, phenyl, and hydrogen derivatives may begin with a Marshall coupling of 1 with a propyne moiety containing an ethyl or hydrogen at the terminal position. In various embodiments, installation of the benzyl and phenyl groups may be achieved using a Pd-catalyzed coupling of the protected alcohol, which then may yield compounds 4, 10, 11, and 12 (e.g., compound 4 and analogues of compound 4 from Scheme A). In various embodiments, transformations that are analogous to those described for Scheme A may be used to generate the corresponding TSA analogues, as illustrated below in Scheme B. In various embodiments, straightforward modifications to Scheme B may be employed to yield other TSA analogues.

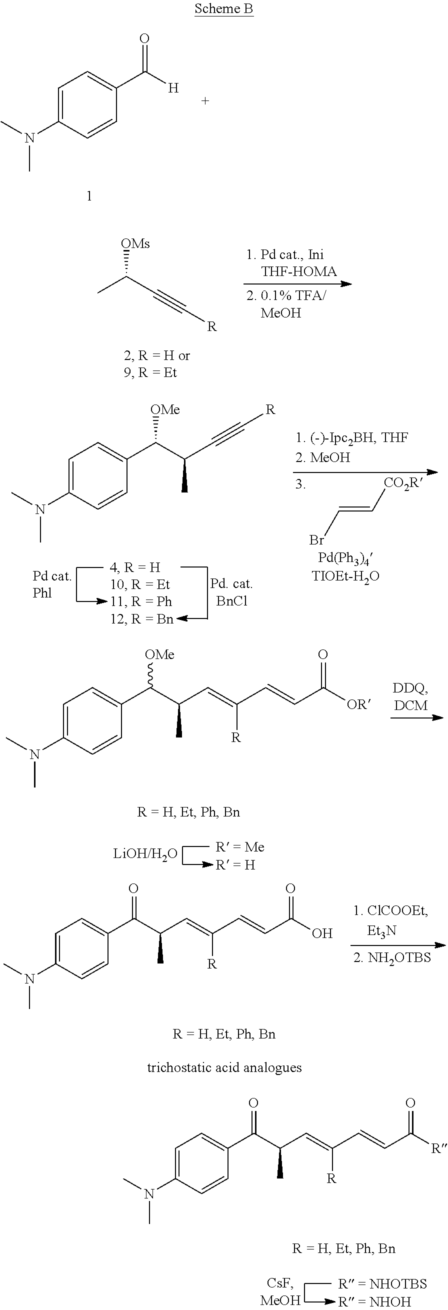

In specific, non-limiting examples, the methods illustrated in Scheme B may be carried out essentially as described for Scheme A, but with the following modifications:

Trichostatin Analogues (R═H, Et, Ph, Bn).

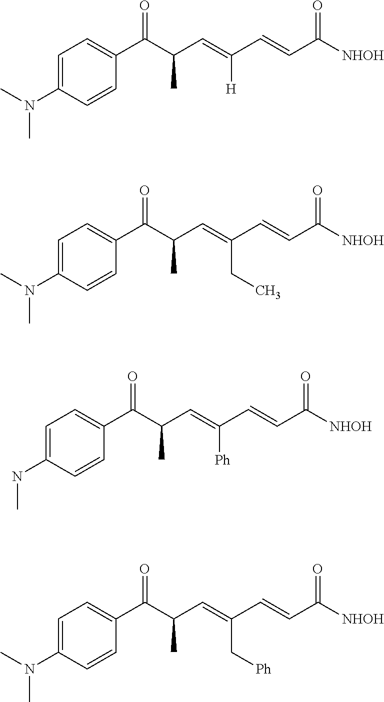

In various embodiments, the analogue with R═H may be obtained by using compound 4 directly in the synthesis without first effecting the conversion to compound 5 that was employed in Scheme A.

In various embodiments, the analogue with R=Et may be obtained by employing the modified propargyl mesylate 9 in place of the propargyl mesylate 2 that was employed in Scheme A.

In various embodiments, the analogue with R=Ph may be obtained by effecting a palladium-catalyzed coupling reaction of the terminal alkyne in compound 4 with iodobenzene to give compound II, which may then be carried through the same sequence of reactions as employed in Scheme A.

In various embodiments, the analogue with R=Bn may be obtained by effecting a palladium-catalyzed coupling reaction of the terminal alkyne in compound 4 with benzyl chloride to give compound 12, which may then be carried through the same sequence of reactions as employed in Scheme A.

In other embodiments illustrated below in Scheme C, the method of Schemes A and B may be further shortened by one step. In various embodiments, the alcohol (14, which is analogous to 3 in Scheme A) obtained by the Marshall reaction in Scheme A may be oxidized. The resulting ketone may then be used for the palladium-catalyzed Suzuki-Miyaura coupling with (E)-methyl 3-bromoacrylate to obtain the methyl trichostatic ester (15). In some embodiments, the methyl ester may then be hydrolyzed to produce trichostatic acid through use of Me₃SnOH, pig liver esterase, or other methods known to those of skill in the art. This four-step strategy to generate trichostatic acid is shorter than all known methods of synthesis for this compound, and TSA may be derived therefrom as described above or by other methods known to those of skill in the art.

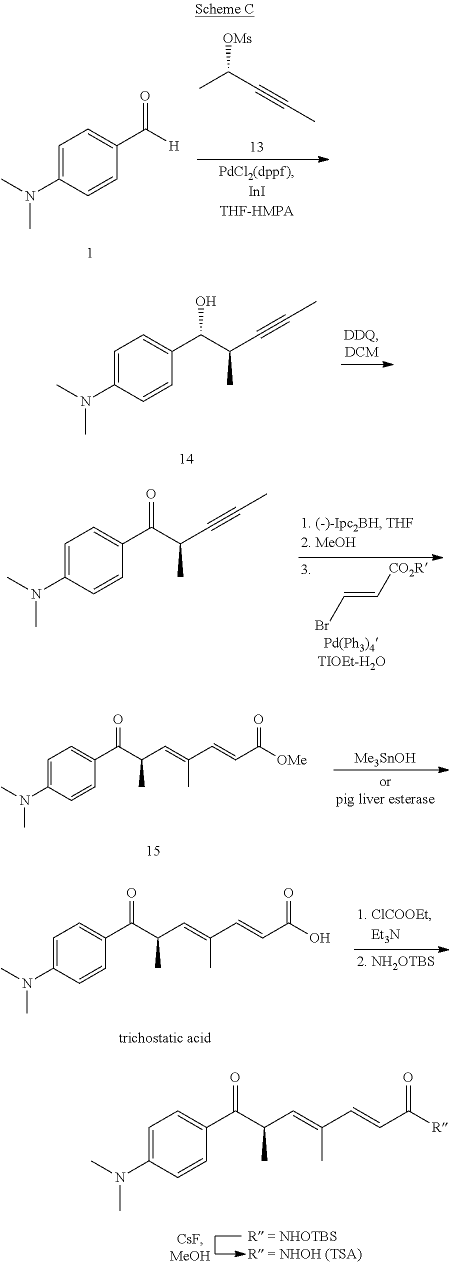

In specific, non-limiting examples, the methods illustrated in Scheme C may be carried out essentially as described for Scheme A, but with the following modifications:

In some embodiments, the reaction sequence in Scheme A may be modified in two key ways: (1) the propargyl mesylate 13 may be used in place of the propargyl mesylate 2; and (2) the resulting product 14 may be oxidized with DDQ directly instead of effecting the oxidation of the corresponding methyl ether at a later stage as shown for conversion of compound 7 to trichostatic acid in Scheme A.

Other embodiments make use of a convergent synthesis starting from methyl methacrylate, as shown below in Scheme D. In some embodiments, transformation may lead to a major subunit as the dienyl bromide 16 containing an ester group as depicted or alternatively a hydroxamic derivative in analogy with the use of 8 in Scheme A. The coupling of this fragment with the ketone 17 may then be carried out using a modified Hartwig-Buchwald-type enolate coupling in the presence of a chiral metal catalyst containing palladium, nickel, or other suitable metals. In various embodiments, the coupling product 15 may be a trichostatic acid ester which by transformation to trichostatic acid allows merging with the method illustrated in Scheme A. In various embodiments, Scheme D may provide the desired product in fewer steps than other known methods.

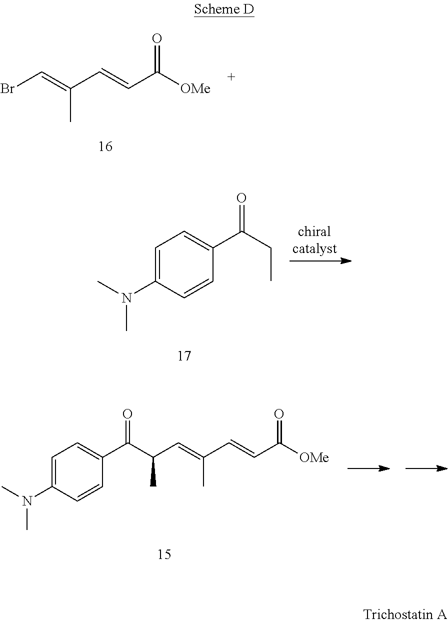

In specific, non-limiting examples, the methods illustrated in Scheme D may be carried out as follows:

### (2E,4E)-methyl 7-(4-(dimethylamino)phenyl)-4,6-dimethyl-7-oxohepta-2,4-dienoate (15)

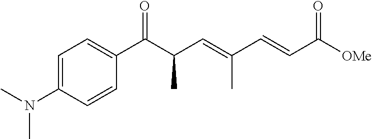

n-Butyllithium (0.15 mL, 0.366 mmol, 2.5 M solution in hexane) was added via syringe to a flame dried flask under argon. The flask was cooled to −78° C. and THF (0.6 mL) was added via syringe. Diisopropylamine (0.05 mL, 0.366 mmol) was added via syringe and the mixture was stirred for 30 minutes. The ketone (65 mg, 0.366 mmol), dissolved in THF (0.6 mL), was added dropwise over the course of 5 minutes at −78° C. The mixture was stirred at this temperature for 30 minutes and then warmed to 0° C. ZnCl₂ (50 mg, 0.366 mmol) was added and the mixture was warmed to RT and stirred for a further 20 minutes. Finally, the Zn-enolate solution was transferred to a flask containing the vinyl halide (50 mg, 0.244 mmol), Ni(cod)₂ (6.7 mg, 0.024 mmol, 10 mol %), and Q-phos (40 mg, 0.054 mmol, 22 mol %). The mixture was stirred at 22° C. TLC analysis (30% Et₂O in hexane) showed the reaction to stop forming product after 3 hours. The mixture was quenched with satd NH₄Cl and extracted with Et₂O. The combined organic extracts were dried with MgSO₄, filtered, and concentrated. Crude ¹H NMR showed product peaks along with unreacted ketone, starting vinyl halide, and some other products. The material was purified by column chromatography (gradient of 30% Et₂O in hexane to 40% Et₂O in hexane) to furnish 24 mg (33%) of 15. ¹H NMR (600 MHz, CDCl₃) δ 7.85 (d, J=8.4 Hz, 2H), 7.31 (d, J=16.2 Hz, 1H), 6.64 (d, J=8.4 Hz, 2H), 6.04 (d, J=9.6 Hz, 1H), 5.84 (d, J=15.6 Hz, 1H), 4.38 (dq, J=9.6, 2.4 Hz, 1H), 3.72 (s, 3H), 3.05 (s, 6H), 1.91 (d, J=2.6 Hz, 3H), 1.31 (d, J=6.6 Hz, 3H); ¹³C NMR (150 MHz, CDCl₃) δ 198.6, 167.9, 153.7, 149.5, 142.1, 132.8, 130.8, 124.0, 116.5, 110.9, 51.7, 41.0, 40.2, 17.9, 12.7; HRMS (ESI) calcd for C₁₈H₂₃NO₃ [M+H]⁺ 302.1751. found 302.1768.

In other embodiments, the method may include a direct coupling reaction of a dienyl bromide 17 with a ketone 18 under modified Negishi cross-coupling conditions, employing a nickel or palladium catalyst with (C₅Ph₅)Fe(C₅H₄PPh₂), to produce compound 19, as shown below in Scheme E. In some embodiments, this method may provide racemic material. In various embodiments, the coupling product 20 may be a trichostatic acid ester, which is a direct precursor of TSA.

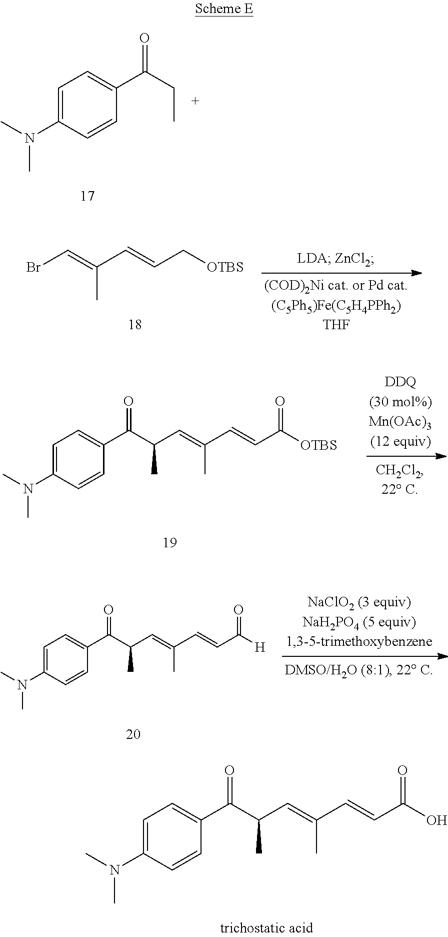

In specific, non-limiting examples, the methods illustrated in Scheme D may be carried out as follows:

### (3E,5E)-7-(tert-Butyldimethylsilyloxy)-1-(4-(dimethylamino)phenyl)-2,4-dimethylhepta-3,5-dien-1-one (19)

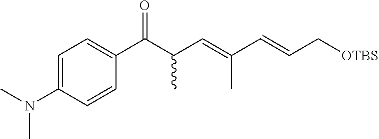

In a flame-dried flask cooled to 0° C. under argon, 2,2,6,6-tetramethylpiperidine (0.093 mL, 0.55 mmol) was charged into a flask and dissolved in THF (1.0 mL). n-BuLi (0.22 mL, 0.55 mmol, 2.5 M soln in hexane) was added via syringe. The solution was stirred for 10 minutes and then cooled to −78° C. Ketone 17 (91 mg, 0.515 mmol), dissolved in THF (0.5 mL), was added dropwise via syringe. The solution was stirred for 1 hour at −78° C. and then warmed to 22° C. The Li-enolate solution was transferred via syringe to a separate flame-dried flask containing ZnCl₂ (84 mg, 0.62 mmol). The mixture was stirred at 22° C. for 30 minutes and then was transferred via syringe to a third flame-dried flask containing Pd(dba)₂ (7.9 mg, 0.014 mmol, 4 mol %), dtbpf (8.1 mg, 0.017 mmol, 5 mol %), and dienyl bromide 18 (100 mg, 0.34 mmol) in THF (0.5 mL). The dark colored solution was stirred under argon at 22° C. and monitored by TLC analysis (30% EtOAc in hexane). After 1 hour, complete consumption of the vinyl halide was observed. The reaction was quenched by the addition of Et₂O (5 mL) and 50% saturated Rochelle's salt solution. The mixture was vigorously stirred for 30 minutes and the layers were separated. The aqueous phase was extracted with Et₂O and the combined organic layers were dried with MgSO₄, filtered, and concentrated. The crude material was purified by column chromatography (10% EtOAc in hexane) to furnish 123 mg (92%) of 19 as an oil that solidified upon refrigeration. mp=38-40° C. ¹H NMR (600 MHz, CDCl₃) δ 7.86 (d, J=9.6 Hz, 2H), 6.63 (d, J=9.6 Hz, 2H), 6.20 (d, J=16.2 Hz, 1H), 5.71-5.66 (m, 1H), 5.56 (d, J=9.6 Hz, 1H), 4.37-4.32 (m, 1H), 4.21 (d, J=6.6 Hz, 2H), 3.05 (s, 6H), 1.89 (d, J=1.2 Hz, 3H), 1.26 (d, J=6.6 Hz, 3H), 0.89 (s, 9H), 0.06 (s, 6H); ¹³C NMR (150 MHz, CDCl₃) δ 199.8, 153.5, 134.6, 133.4, 133.0, 130.8, 127.2, 124.5, 110.9, 64.2, 40.6, 40.2, 26.2, 18.7, 18.0, 13.0, −4.9; HRMS (ESI) calcd for C₂₃H₃₇NO₂Si [M+H]⁺ 388.2666. found 388.2665.

### (2E,4E)-7-(4-(Dimethylamino)phenyl)-4,6-dimethyl-7-oxohepta-2,4-dienal (20)

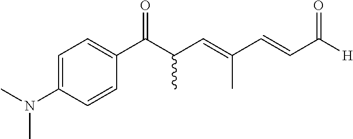

The cross-coupled product 19 (28 mg, 0.072 mmol) was dissolved in DCM (0.5 mL) and stirred at 22° C. Mn(OAc)₃ (116 mg, 0.433 mmol) was added as a single portion, followed by the dropwise addition of DDQ (0.14 mL, 0.014 mmol, 0.1 M solution in DCM) via syringe. The mixture was vigorously stirred for 2 hours, and TLC analysis (30% EtOAc in hexane) indicated that the reaction had halted, but was not complete (approximately 50% conversion). Mn(OAc)₃ (116 mg, 0.433 mmol) was added again and the reaction proceeded. After 8 hours, the reaction was filtered through Celite and concentrated. The crude material was purified by column chromatography (20% EtOAc in hexane) to furnish 16.6 mg (85%) of 20 as a light-yellow oil. ¹H NMR (600 MHz, CDCl₃) δ 9.54 (d, J=7.8 Hz, 1H), 7.85 (d, J=9.0 Hz, 2H), 7.11 (d, J=15.6 Hz, 1H), 6.65 (d, J=9.0 Hz, 2H), 6.19 (d, J=9.6 Hz, 1H), 6.15-6.11 (dd, J=15.6, 7.8 Hz, 1H), 4.46-4.40 (m, 1H), 3.06 (s, 6H), 1.95 (d, J=1.2 Hz, 3H), 1.33 (d, J=7.2 Hz, 3H); ¹³C NMR (150 MHz, CDCl₃) δ 197.1, 193.1, 156.3, 152.5, 143.0, 132.0, 129.6, 126.6, 122.6, 109.7, 39.8, 38.9, 16.9, 11.6; HRMS (ESI) calcd for C₁₇H₂₁NO₂[M+H]⁺ 272.1645. found 272.1632.

### Trichostatic Acid

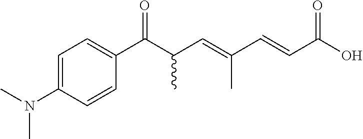

To a solution of aldehyde 20 (33 mg, 0.122 mmol) in DMSO (1.2 mL), 1,3,5-trimethoxybenzene (41 mg, 0.243 mmol) was added as a solid. The mixture was stirred for 5 minutes and then NaClO₂ (41 mg, 0.365 mmol, 80% technical grade) and NaH₂PO₄ (73 mg, 0.608 mmol), dissolved in water (0.2 mL), was added dropwise via pipette. The mixture was vigorously stirred and monitored by TLC analysis (50% EtOAc in hexane, 0.1% HOAc). After 4 hours, the aldehyde was completely consumed. The reaction was quenched with 50% Na₂S₂O₃ and acidified with 1 N HCl (3 mL). The mixture was extracted with EtOAc (3×10 mL) and the combined organic extracts were washed with water (5×20 mL). The organics were dried with MgSO₄, filtered, and concentrated. The crude material was purified by column chromatography (40% EtOAc in hexane, 0.1% HOAc), furnishing 27 mg (76%) of the trichostatic acid as a light yellow oil. ¹H NMR (600 MHz, CDCl₃) δ 7.85 (d, J=9.6 Hz, 2H), 7.37 (d, J=15.6 Hz, 1H), 6.64 (d, J=9.6 Hz, 2H), 6.10 (d, J=10.2 Hz, 1H), 5.82 (d, J=15.6 Hz, 1H), 4.39 (dq, J=7.2, 2.4 Hz, 1H), 3.06 (s, 6H), 1.92 (d, J=1.2 Hz, 3H), 1.31 (d, J=7.2 Hz, 3H); ¹³C NMR (150 MHz, CDCl₃) δ 198.5, 172.6, 153.7, 151.7, 143.3, 132.8, 130.9, 123.9, 115.9, 110.9, 41.0, 40.2, 17.9, 12.7.

In various embodiments, Schemes A-E all provide convergent routes that include a small number of steps. Additionally, Schemes A-E all make use of readily available, inexpensive starting materials, and efficient catalytic processes may be used to fix the chirality in the syntheses, which may be important for use on industrial scales. Furthermore, these routes of synthesis make possible the synthesis of a number of novel TSA analogues in an efficient manner, and also may provide a very short path for preparation of TSA and TSA analogues.

Although certain embodiments have been illustrated and described herein, it will be appreciated by those of ordinary skill in the art that a wide variety of alternate and/or equivalent embodiments or implementations calculated to achieve the same purposes may be substituted for the embodiments shown and described without departing from the scope. Those with skill in the art will readily appreciate that embodiments may be implemented in a very wide variety of ways. This application is intended to cover any adaptations or variations of the embodiments discussed herein. Therefore, it is manifestly intended that embodiments be limited only by the claims and the equivalents thereof.

What is claimed is:

1. A method of synthesizing TSA or a TSA analogue, the method comprising:
providing (2E,4E)-methyl 7-methoxy-4,6-dimethyl-7-(4-dimethylaminophenyl)hepta-2,4-dienoate: 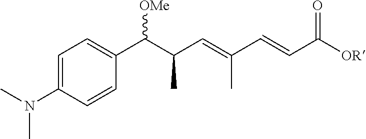
wherein R′=Me;
reacting the (2E,4E)-methyl 7-methoxy-4,6-dimethyl-7-(4-dimethylaminophenyl)hepta-2,4-dienoate with LiOH/H₂O to replace the Me at R′ with H to form (2E,4E)-methyl 7-methoxy-4,6-dimethyl-7-(4-dimethylaminophenyl)hepta-2,4-dienoic acid;
performing an oxidation reaction on the (2E,4E)-methyl 7-methoxy-4,6-dimethyl-7-(4-dimethylaminophenyl)hepta-2,4-dienoic acid with 2,3-dichloro-5,6-dicyano-1,4-benzoquinone (DDQ) and dichloromethane (DCM) to form trichostatic acid; and
reacting the trichostatic acid with hydroxylamine or a hydroxylamine derivative to form the TSA or TSA analogue.

2. The method of claim 1, wherein reacting the trichostatic acid to form the TSA or TSA analogue comprises:
reacting the trichostatic acid with CICOOEt, TEA, and NH₂OTBS to form compound A: 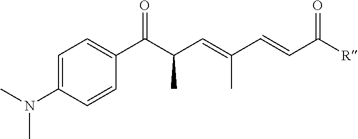
wherein R″═NHOTBS; and
reacting compound A with CsF and MeOH to form the TSA or TSA analogue.

3. The method of claim 1, wherein the step of providing (2E,4E)-methyl 7-methoxy-4,6-dimethyl-7-(4-dimethylaminophenyl)hepta-2,4-dienoate comprises:
providing (1R,2R)-1-[4-(dimethylamino)phenyl]-2-methylpent-3-yn-1-ol, wherein R═OH;
reacting the (1R,2R)-1-[4-(dimethylamino)phenyl]-2-methylpent-3-yn-1-ol with 0.1% trifluoroacetic acid (TFA)/MeOH to replace the OH at R with OMe to form (R)-5-methoxy-5-[4-(dimethylamino)phenyl]-4-methyl-3-pentyne; and
performing a palladium-catalyzed Suzuki-Miyaura coupling on (R)-5-methoxy-5-[4-(dimethylamino)phenyl]-4-methyl-3-pentyne with (−)-Ipc₂BH₂THF, MeOH, Pd(Ph₃)₄, TIOEt-H₂O, and (E)-methyl 3-bromopropenoate to form the (2E,4E)-methyl 7-methoxy-4,6-dimethyl-7-(4-dimethylaminophenyl)hepta-2,4-dienoate.

4. The method of claim 3, wherein providing the (1R,2R)-1-[4-(dimethylamino)phenyl]-2-methylpent-3-yn-1-ol comprises:
providing 4-(dimethylamino)benzaldehyde; and
performing a palladium-catalyzed Marshall coupling of the 4-(dimethylamino)benzaldehyde with a chiral mesylate containing a propyne moiety in the presence of Pd-cat, Inl, and THF-HMPA, thus forming the (1R,2R)-1-[4-(dimethylamino)phenyl]-2-methylpent-3-yn-1-ol.

5. The method of claim 4, wherein the chiral mesylate containing a propyne moiety is:

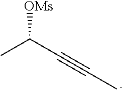

6. A method of synthesizing a TSA analogue, the method comprising:
providing compound B, wherein R═H, Et, Ph, or Bn: 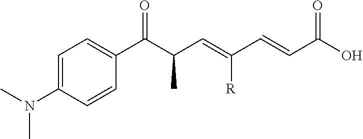
reacting compound B with CICOOEt, TEA, and NH₂OTBS to form compound C: 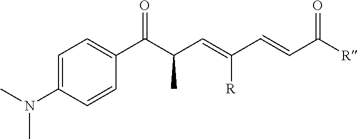
wherein R″═NHOTBS; and
reacting compound C with CsF and MeOH to convert the NHOTBS at R″ with NHOH, thereby synthesizing the TSA analogue.

7. The method of claim 6, wherein the step of providing compound B comprises:
providing compound D: 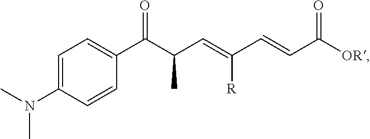
wherein R′=Me;
reacting compound D with LiOH/H₂O to replace the Me at R′ with H to form compound E; and
reacting compound E with 2,3-dichloro-5,6-dicyano-1,4-benzoquinone (DDQ) and dichloromethane (DCM) to form compound B.

8. The method of claim 7, wherein the step of providing compound D comprises:
providing (R)-1-methoxy-1-[4-(dimethylamino)phenyl]-2-methyl-3-hexyne or (R)-4-methoxy-4-[4-(dimethylamino)phenyl]-4-methyl-1-butyne; and
performing a hydroboration and palladium-catalyzed Suzuki-Miyaura coupling reaction with the (R)-1-methoxy-1-[4-(dimethylamino)phenyl]-2-methyl-3-hexyne or (R)-4-methoxy-4-[4-(dimethylamino)phenyl]-4-methyl-1-butyne using (−)-Ipc₂BH₂.THF, MeOH, Pd(Ph₃)₄, and TIOEt-H₂O, and (E)-methyl 3-bromopropenoate to form compound D.

9. The method of claim 8, wherein in the (R)-1-methoxy-1-[4-(dimethylamino)phenyl]-2-methyl-3-hexyne or (R)-4-methoxy-4-[4-(dimethylamino)phenyl]-4-methyl-1-butyne, R═H; and wherein prior to performing the palladium-catalyzed Suzuki-Miyaura coupling, the method further comprises reacting the (R)-1-methoxy-1-[4-(dimethylamino)phenyl]-2-methyl-3-hexyne or (R)-4-methoxy-4-[4-(dimethylamino)phenyl]-4-methyl-1-butyne with palladium catalyst and BnCl to replace the H at position R with Bn.

10. The method of claim 8, wherein in the (R)-1-methoxy-1-[4-(dimethylamino)phenyl]-2-methyl-3-hexyne or (R)-4-methoxy-4-[4-(dimethylamino)phenyl]-4-methyl-1-butyne R═H; and wherein prior to performing the palladium-catalyzed Suzuki-Miyaura coupling, the method further comprises reacting compound L with palladium catalyst and iodobenzene to replace the H at position R with Ph.

11. The method of claim 8, wherein the step of providing the (R)-1-methoxy-1-[4-(dimethylamino)phenyl]-2-methyl-3-hexyne or (R)-4-methoxy-4-[4-(dimethylamino)phenyl]-4-methyl-1-butyne comprises:
providing 4-(dimethylamino)benzaldehyde;
performing a palladium-catalyzed Marshall coupling of the 4-(dimethylamino)benzaldehyde with compound F: 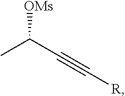
wherein R=Et, H, (S)-2-methanesulfonoxy-3-hexyne, or (S)-3-methanesulfonoxy-1-butyne, and wherein the coupling is carried out in the presence of palladium catalyst, Inl, THF-HMPA, 0.1% TFA/MeOH, thus forming the (R)-1-methoxy-1-[4-(dimethylamino)phenyl]-2-methyl-3-hexyne or (R)-4-methoxy-4-[4-(dimethylamino)phenyl]-4-methyl-1-butyne.

12. A method of synthesizing TSA or a TSA analogue, the method comprising:
providing (2E,4E,6R)-methyl 4,6-dimethyl-7-(4-dimethylaminophenyl)-7-oxohepta-2,4-dienoate;
reacting the (2E,4E,6R)-methyl 4,6-dimethyl-7-(4-dimethylaminophenyl)-7-oxohepta-2,4-dienoate with Me₃SnOH or pig liver esterase to form trichostatic acid; and
reacting the trichostatic acid with hydroxylamine or a hydroxylamine derivative to form the TSA or TSA analogue.

13. The method of claim 12, wherein reacting the trichostatic acid to form the TSA or TSA analogue comprises:
reacting the trichostatic acid with CICOOEt, TEA, and NH₂OTBS to form compound A: 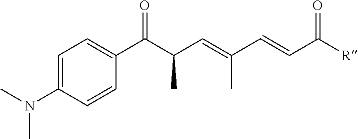
wherein R″═NHOTBS; and
reacting compound A with CsF and MeOH to form TSA or a TSA analogue.

14. The method of claim 13, wherein the step of providing the (2E,4E,6R)-methyl 4,6-dimethyl-7-(4-dimethylaminophenyl)-7-oxohepta-2,4-dienoate comprises:
providing 2-methyl-1-[4-(dimethylamino)phenyl]pent-3-yne-1-one, and
performing a hydroboration and palladium-catalyzed Suzuki-Miyaura coupling on the 2-methyl-1-[4-(dimethylamino)phenyl]pent-3-yne-1-one with (−)-Ipc₂BH, THF, MeOH, (E)-methyl 3-bromopropenoate, Pd(Ph₃)₄, and TIOEt-H₂O to form the (2E,4E,6R)-methyl 4,6-dimethyl-7-(4-dimethylaminophenyl)-7-oxohepta-2,4-dienoate.

15. The method of claim 14, wherein the step of providing the 2-methyl-1-[4-(dimethylamino)phenyl]pent-3-yne-1-one comprises:
providing (1R,2R)-2-methyl-1-[4-(dimethylamino)phenyl]pent-3-yne-1-ol, wherein R═OH, and
reacting the (1R,2R)-2-methyl-1-[4-(dimethylamino)phenyl]pent-3-yne-1-ol with 2,3-dichloro-5,6-dicyano-1,4-benzoquinone (DDQ) and dichloromethane (DCM) to form 2-methyl-1-[4-(dimethylamino)phenyl]pent-3-yne-1-one.

16. The method of claim 15, wherein the step of providing the (1R,2R)-2-methyl-1-[4-(dimethylamino)phenyl]pent-3-yne-1-ol comprises:
providing 4-(dimethylamino)benzaldehyde, and
performing a palladium-catalyzed Marshall coupling of the 4-(dimethylamino)benzaldehyde with a chiral mesylate containing a propyne moiety in the presence of palladium catalyst, Inl, and THF-HMPA, thus forming the (1R,2R)-2-methyl-1-[4-(dimethylamino)phenyl]pent-3-yne-1-ol.

17. The method of claim 16, wherein the chiral mesylate containing a propyne moiety is:

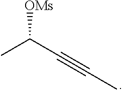

18. The method of claim 12, wherein the step of providing the (2E,4E,6R)-methyl 4,6-dimethyl-7-(4-dimethylaminophenyl)-7-oxohepta-2,4-dienoate comprises coupling methyl (2E,4E)-5-bromo-4-methylpenta-2,4-dienoate with 4-(dimethylamino)propiophenone using a Hartwig-Buchwald-type enolate coupling in the presence of a chiral metal catalyst comprising palladium or nickel.

19. A method of synthesizing TSA or a TSA analogue, the method comprising:
providing 4-(dimethylamino)propiophenone and methyl (2E,4E)-5-bromo-4-methylpenta-2,4-dienoate; and
coupling the 4-(dimethylamino)propiophenone and the methyl (2E,4E)-5-bromo-4-methylpenta-2,4-dienoate under modified Negishi cross-coupling conditions, wherein the coupling reaction employs a nickel catalyst and ligand G: 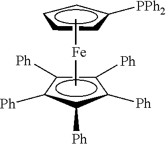
wherein the coupling reaction produces racemic (2E,4E)-methyl 4,6-dimethyl-7-(4-dimethylaminophenyl)-7-oxohepta-2,4-dienoate, and
reacting the racemic (2E,4E)-methyl 4,6-dimethyl-7-(4-dimethylaminophenyl)-7-oxohepta-2,4-dienoate with hydroxylamine or a hydroxylamine derivative to form the TSA or TSA analogue.

20. A method of synthesizing a trichostatic acid analogue, the method comprising:
providing compound H having the structure: 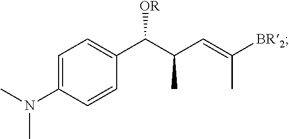
and
reacting the compound H with compound I having the structure: 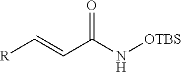
wherein R=iodide, bromide, tosylate, or para-toluenesulfonate.
